(19) United States
(12) Patent Application Publication
(10) Pub. No.: US 2019/0343076 A1
(43) Pub. Date: Nov. 14, 2019

(54) APPARATUS, SYSTEM AND METHOD FOR MONITORING AND/OR CONTROLLING AN ANIMAL

(71) Applicant: Peiker Holding GmbH, Bad Homburg (DE)

(72) Inventors: Daniela BÜDENBENDER, Liederbach (DE); Christoph SCHILLO, Friedrichsdorf (DE)

(73) Assignee: Peiker Holding GmbH, Bad Homburg (DE)

(21) Appl. No.: 16/408,872

(22) Filed: May 10, 2019

(30) Foreign Application Priority Data
May 14, 2018 (DE) 10 2018 111 467.2

(51) Int. Cl. A01K 15/02; G08B 21/02; G06F 3/16; A01K 29/00; A01K 11/00; A01K 7/02; A61B 5/0205

(52) U.S. Cl. CPC A01K 15/02; G08B 21/02; G06F 3/165; A61B 5/015; A01K 11/006; A01K 7/02; A61B 5/02055; A01K 29/005

(57) ABSTRACT

The present invention relates to an apparatus for monitoring and/or controlling an animal, in particular a horse, by repeatedly recording at least one vital function and/or a movement behavior of the animal.

### FIELD OF THE INVENTION

The present invention relates to an apparatus, a system and a method for monitoring and/or controlling an animal.

### BACKGROUND OF THE INVENTION

DE 20 2013 008 719 U1 discloses a system for playing sound waves to animals and thus promoting relaxation, recovery and well-being.

### SUMMARY OF THE INVENTION

The object of the present invention is to propose an apparatus, a system or a method, by means of which the individual needs of the animal can be addressed automatically. In addition, an object of the present invention is also, in particular, to monitor the animal and increase the safety of the animal.

The apparatus according to the present invention for monitoring and/or controlling an animal, especially a horse, by repeatedly recording at least one vital function and/or a movement behavior of the animal comprises means for storage and/or reproduction of at least one audio file, as well as at least one sensor and/or at least one interface for a sensor, as well as means for recording and evaluating measurements from the at least one sensor and/or the at least one interface and/or at least one interface for a component from the group of audio devices, video devices, communication devices or data recording devices. By the apparatus being equipped with means for recording and evaluation of measurements from the at least one sensor and/or from the at least one interface for a sensor, it is possible to control the means for storage and/or reproduction of at least one audio file depending on the one or more sensors that monitor the animal, and therefore play audio files as required.

It is further provided that the apparatus comprises a memory as a storage means, in which, in particular, audio files are stored, that the apparatus comprises a playback device and preferably an acoustic output device for outputting individual audio files as the reproduction means, that as a recording means the apparatus comprises a recording device with the at least one sensor or the least one interface for a sensor for recording measurements of the at least one vital function of the animal and/or with the at least one sensor or the at least one interface for a sensor for recording measurements of the movement behavior of the animal, wherein as an evaluation means the apparatus has a program-controlled processor with a memory, wherein in the memory mappings are stored, by means of which specific audio files and/or control instructions are assigned to specific measurements or measurement combinations, and that the program-controlled processor activates the playback device by means of defined control commands for playing back audio files determined from the mappings. By means of an apparatus with which an alternative or cumulative recording of vital function or movement behavior of the animal and the program-controlled evaluation and selection of audio files is carried out, it is possible to individually sonicate the animal and act upon it in a targeted manner.

The present invention also relates to an apparatus which is designed such that the apparatus comprises a memory as a storage means, in which, in particular, audio files are stored, that the apparatus comprises a playback device and preferably an acoustic output device for outputting individual audio files as the reproduction means, that as a recording means the apparatus comprises a recording device with the at least one sensor or the least one interface for a sensor for recording measurements of the at least one vital function of the animal and/or with the at least one sensor or the at least one interface for a sensor for recording measurements of the movement behavior of the animal, that as an evaluation means the apparatus comprises a program-controlled processor with a memory, wherein in the memory mappings are stored, by means of which specific audio files and/or control instructions are assigned to specific measurements or measurement combinations, and that the program-controlled processor activates the playback device by means of defined control commands for playing back audio files determined using the mappings. By means of an apparatus, with which an alternative or cumulative recording of vital function or movement behavior of the animal and the program-controlled evaluation and selection of audio files is carried out, it is possible to individually sonicate the animal and act upon it in a targeted manner.

It is also provided that by means of the mappings, measurements or measurement combinations are assigned to loudness specifications as control instructions. It is thus possible to adapt this audio file in terms of loudness to the identified state of health or mental state of the animal.

It is also provided that the sensor or sensors of the recording device that are used for recording measurements of the at least one vital function is/are implemented as a heart rate sensor and/or respiration frequency sensor and/or body temperature sensor, such as a thermal image sensor and/or a moisture sensor for detecting sweat and/or a blood pressure sensor and/or thermal imaging sensor and/or acoustic sensor, such as a microphone and/or an acceleration sensor for detecting shivering of the animal, and/or that the sensor or sensors of the recording device which are used for recording measurements of the movement behavior is/are implemented as an image sensor, such as a CCD sensor or thermal imaging sensor, in order to be able to capture movement even in the dark, and/or as a motion sensor, in particular as an ultrasound sensor and/or as an acceleration sensor. By means of such sensors, a plurality of vital functions as well as the movement behavior can be recorded in a wide variety of ways. Individual sensors, such as accelerometers, can also be used to capture both a vital function and a movement behavior. One example that could be mentioned here is a thermal image sensor, which can be used to capture both the body temperature as well as movements of the animal, especially during the hours of darkness.

It is also provided that the apparatus comprises at least one network module as an interface for the wireless connection of at least one of the sensors and/or for wireless integration of the apparatus into a wireless network, such as, in particular, a WLAN wireless network and/or a BlueTooth wireless network and/or a cellular network, wherein it is provided, in particular, that the wirelessly connected sensor is connected to the animal, in particular, by means of a belt or is implanted in the animal and/or

that as an interface the apparatus comprises at least one network module for the wired connection of at least one of the sensors and/or the wired integration of the device into a wired network, such as, in particular, a local area network or a CAN bus network. In this way, the number and type of the sensors can be adjusted individually and straightforwardly. The apparatus can, therefore, also be retrofitted with new sensors.

It is also provided that the output device includes at least one speaker, wherein the output device, in particular, comprises at least two speakers, wherein the speakers have adjacent frequency spectra and/or partially overlapping frequency spectra and/or identical frequency spectra, that it is also provided, in particular, that the output device comprises at least one contact loudspeaker which is matched to a low frequency spectrum and/or that the output device comprises at least one tweeter which is matched to a high frequency spectrum, in particular above 20 kHz, and that it is also provided, in particular, that at least one of the speakers is designed as an active speaker which is wirelessly connected to the playback device. Such a design of the output device means that the output device can be individually matched to the frequency spectrum detectable from the respective animal, so that the effect is optimized.

It is further provided that the recording device comprises at least one additional sensor for detecting a person approaching the animal or detecting a person present in the vicinity of the animal, wherein the recording device preferably comprises an image sensor, in particular a CCD sensor or a thermal image sensor and/or a motion sensor, in particular, an ultrasound sensor and/or a microphone and/or a vibration sensor, wherein the software-controlled processor is, in particular, programmed in such a way that, in particular, using face recognition and/or gesture recognition, it can distinguish between people with access authorization and people without access permission and that the apparatus comprises an alarm system, wherein the alarm system comprises, in particular, an optical and/or acoustic alarm output unit connected to a wired or wireless network. This allows animals and, in particular, horses, which are accommodated, for example, in easily accessible stalls, to be protected against unwanted doping or against unwanted feeding. At the same time, authorized people can feed and care for the animal without having to handle or carry keys or other means of access authorization with them.

It is also provided that the recording device comprises at least one additional sensor for recording a function of technical devices of an animal stall, in particular, a horse stable, wherein one of the additional sensors is implemented, in particular, as a mass flow sensor for a supply pipe of a drinking trough and/or wherein one of the additional sensors is implemented, in particular, as a monitoring sensor for a door or a window or a feeding flap. This makes it possible to determine, for example, whether the animal is drinking at the usual intervals and/or in the usual quantities and if not, it can be assumed that the trough is contaminated in some way, which prevents the animal from drinking. This can also allow actions of a person to be monitored and, as a result, for example, conclusions to be drawn as to the intentions of a non-authorized person, so that, for example, differently conditioned alarms are triggered.

It is further provided that at least one of the audio files comprises sounds produced by animals, in particular, by horses, and preferably grazing sounds of ungulates, in particular, horses. By using such audio files it is possible, in particular, to calm an ungulate or horse.

Finally, it is also provided that as an evaluation means the apparatus comprises a program-controlled processor with a memory and, in particular, at least one additional sensor that generates measurements for environmental parameters, wherein in the memory mappings are stored, in which specific control instructions are assigned to specific measurements or measurement combinations, and that the apparatus (1) comprises a control device and that by means of defined control commands of the program-controlled processor, devices for influencing environmental parameters are controlled, wherein the devices are implemented, in particular, as a lighting device and/or as a fragrance dispenser device and/or a cooling device and/or a heating device. This means it is possible to influence the well-being of a horse by controlling other environmental parameters. In addition, or alternatively, open-loop or closed-loop control of other equipment associated with the stable can also be provided in a time- and/or event-dependent manner.

The system according to the present invention for monitoring and/or controlling at least two animals, in particular, horses, comprises at least two sets of apparatus, wherein the apparatuses are connected to each other via a communication link in such a way that each apparatus has access to the current measurements of the other apparatus or apparatuses and/or to the control commands of the other apparatus or apparatuses for playing individual audio files. This provides the advantage that by networking the devices in a stable, for example, a trend can be detected, such as a spreading state of unrest, and this can be counteracted by preventative measures. Furthermore, by networking the devices a synchronization or coordination of the audio files that are played is also possible, so that a consistent sound picture can be produced in the barn.

The method according to the present invention for monitoring and/or controlling an animal, in particular, a horse, by repeatedly recording at least one vital function and/or a movement behavior of the animal by means of an apparatus comprises the steps:

that audio files are stored in a means for storing audio data (memory),
that using recording means (recording device), which comprise at least one sensor and/or at least one interface for a sensor for recording measurements of the at least one vital function of the animal and/or which comprise at least one sensor and/or at least one interface for a sensor for recording measurements of the movement behavior of the animal, at least one measurement is recorded,
that as an evaluation means the apparatus comprises a program-controlled processor with a memory, wherein in the memory mappings are stored, by means of which one of the stored audio files is identified, which is assigned to a specific measurement or specific measurements, and
that commands defined by the program-controlled processor for playing individual audio files on a playback device are activated with an output device. Because a condition of the animal is detected by sensors and audio files are played on the basis of a program-controlled evaluation of the measured values, it is possible to play back audio files as and when needed.

It is also provided that while an audio file is being played, the current measurements recorded by the recording device are compared by means of the program-controlled processor with older measurements recorded by the recording device, and evaluated by the program-controlled processor as to whether an improvement in the vital functions and/or the movement behavior is obtained by playing one of the audio files,

wherein in the case of a deterioration in the vital functions and/or the movement behavior, a message is generated by the program-controlled processor, which is forwarded via a network module of the device to at least one message receiver,
wherein the message receiver is implemented, in particular, as a mobile phone and/or an alarm device. This will allow the animal to be treated at an early stage and thus with greater chances of success and less expense.

It is also provided that the recording device comprises at least one additional sensor for detecting a person approaching the animal or for detecting a person present in the vicinity of the animal,

wherein the recording device preferably comprises an image sensor (CCD sensor, thermal imaging sensor) and a motion sensor, in particular, an ultrasound sensor,
wherein the software-controlled processor is programmed, in particular, with distinguishing features of persons with access authorization, in such a way that persons with access authorization can be distinguished from persons without access authorization,
wherein whenever a person without access authorization is detected, the software-controlled processor initiates at least one action,
wherein by means of a first action a video recording of activities of the person without access permission is performed and/or wherein by means of a second action an alarm system of the apparatus is activated and/or wherein by means of a third action of the software-controlled processor a message is generated, which is forwarded via a network module of the apparatus to at least one message receiver, wherein the message receiver is implemented, in particular, as a mobile phone and/or alarm output device. By using such a method, easily accessible stalls can also be effectively secured and the access for authorized people is possible without them needing to carry or handle keys or similar means of access.

It is also provided that at least one of the audio files is generated by an audio recording of sounds identical to those occurring in nature, wherein the nature-identical sounds are generated, in particular, in a sound spectrum audible to animals, in particular to horses, and outside a range audible to animals, in particular horses, and wherein the nature-identical sounds are produced, in particular, by at least one animal, in particular an ungulate, and preferably a horse. Such a method can be used to create audio files at low cost.

Furthermore, the method provides that before the sounds of the audio file are played back, they are analyzed into different frequency ranges by means of a spectral decomposition and the reproduction of the different frequency ranges, in particular, sound audible to the animal, infra-sound which is inaudible to the animal and ultrasound inaudible to the animal are reproduced on different output devices or different interfaces for output devices. This allows the audio file to be reproduced in its different frequencies at appropriate places. In particular, the infra-sound can be output, for example, with a contact loudspeaker through the floor of the stall.

Finally, devices for affecting environmental parameters, which are implemented, in particular, as a lighting device and/or as a fragrance dispenser device and/or as a cooling system and/or a heating system, are controlled by the control device on the basis of measurement values. Thus, the environment of the animal can be controlled to a large extent.

### BRIEF DESCRIPTION OF THE INVENTION

Additional details of the present invention are described in the drawing on the basis of exemplary embodiments shown schematically.

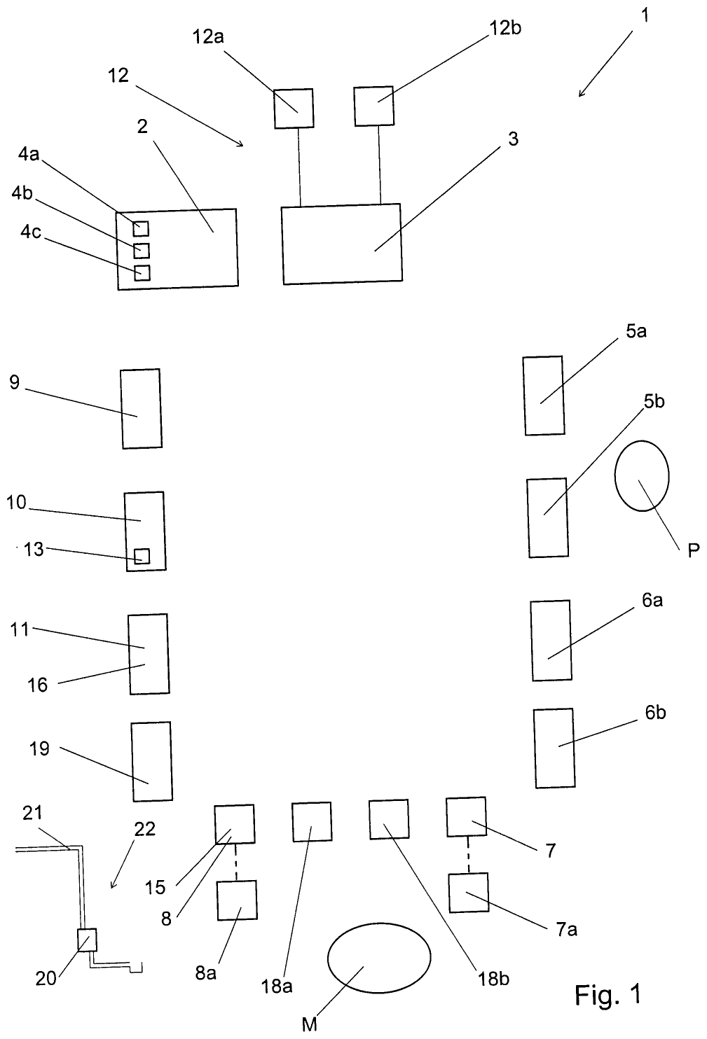

FIG. 1 is a schematic drawing of an apparatus according to the present invention.

### DETAILED DESCRIPTION OF THE INVENTION

FIG. 1 shows an apparatus 1 according to the present invention in a schematic view. The apparatus comprises a memory 2 as a storage means and a playback device 3 as a reproduction means, wherein a plurality of audio files 4a, 4b and 4c are stored in the memory 2. The apparatus 1 further comprises at least one first sensor 5a and a second sensor 5b, and a third sensor 6a and a fourth sensor 6b and, optionally, a first interface 7 for a sensor 7a and a second interface 8 for a sensor 8a. The apparatus also comprises a recording device 9 and a program-controlled processor 10 as means for recording and evaluation of measurements from the sensors or interfaces. Finally, the apparatus also comprises an interface 11 for a component from the group of audio devices, video devices, communication devices or data recording devices.

The playback device 3 comprises an output device 12 for outputting individual audio files 4a, 4b, 4c. With the sensors 5a and 5b, measurements of vital functions are recorded, such as heartbeat and heart rate of a horse P. With the sensor 6a, 6b measurements of a movement behavior are recorded, such as rearing and shivering of the horse P. With the sensor 7a connected to the interface 7, another vital function of the horse P is monitored, wherein the interface 7 is implemented as a wireless interface and the sensor 7a is implemented as a wireless sensor. With the sensor 8a connected to the interface 8 the movement behavior of the horse P is additionally monitored, wherein the interface 8 is implemented as a wireless interface and the sensor 8a is implemented as a wireless sensor.

As an evaluation means the apparatus 1 comprises a program-controlled processor 10 with a memory 13, wherein in the memory 13 mappings are stored, by means of which specific audio files 4a, 4b, 4c and/or control instructions are assigned to specific measurements or measurement combinations detected by the sensors 5a, 5b, 6a, 6b, 7a, 8a. The program-controlled processor 10 activates the playback device 3 by means of defined control commands for playing back audio files 4a or 4b or 4c determined using the mappings. Via the mappings, control instructions in the form of loudness specification are also assigned to individual measurements or combinations of individual measurements. Accordingly, a certain audio file 4a or 4b or 4c is selected for playback depending on the measurements and this is then played back at a specific loudness depending on the measurements.

The interfaces 7, 8 are each implemented by a network module 14, 15 for the wireless connection of at least one of the sensors 7a or 8a.

A further interface 11 is implemented as a network module 16 for the wireless integration of the apparatus in a wireless network 17.

The output device 12 comprises speakers 12a, 12b, wherein the speakers 12a, 12b have adjacent frequency spectra. The output device 12 comprises the contact loudspeaker 12a which is matched to a low frequency spectrum and at the same time the output device 12 comprises a tweeter 12b matched to a high frequency range, in particular above 20 kHz.

The recording device 9 of the apparatus 1 comprises additional sensors 18a, 18b for detecting a person M approaching the horse P, a person M present in the vicinity of the horse P, wherein the sensor 18a is implemented as image sensor 18a and wherein the sensor 18b is implemented as motion sensor 18b. The software-controlled processor 10 is also programmed in such a way that it can be used to distinguish between people with access authorization and people without access authorization by means of face detection and/or gesture recognition. In this case, the apparatus comprises an alarm device 19, with which via an alarm output unit the presence of a person on-site without access permission can be alerted. Alternatively or additionally, it is also provided that an alarm is transmitted using an alarm forwarding unit via the wireless network 17, e.g. to a mobile phone or an alarm center.

The recording device 9 comprises an additional sensor 20, with which a function of a supply pipe 21 of a drinking trough 22 is monitored.

### LIST OF REFERENCE NUMERALS

1 apparatus
2 memory
3 playback device
4a, 4b, 4c, audio file
5a sensor
5b sensor
6a sensor
6b sensor
7 first interface 7 for 7a
7a sensor 7a
8 second interface 8 for 8a
8a sensor 8a
9 recording device
10 processor
11 interface
12 output device
13 memory
14, 15 network module
16 network module
17 wireless network
18a, 18b sensor
19 alarm device
20 sensor
21 supply pipe
22 drinking trough
   M person
   P horse

1. An apparatus for monitoring and/or controlling an animal, by repeatedly recording at least one vital function and/or a movement behavior of the animal, wherein the apparatus comprises:
means for the storage and/or reproduction of at least one audio file, and
at least one sensor and/or at least one interface for a sensor; and
means for recording and evaluating measurements from the at least one sensor and/or the at least one interface and/or
at least one interface for a component selected from the group consisting of audio devices, video devices, communication devices of-and data recording devices.

2. The apparatus as claimed in claim 1,
wherein the apparatus comprises a memory as a storage means, in which audio files are stored,
wherein the apparatus comprises a playback device for outputting individual audio files as the reproduction means,
wherein as a recording means the apparatus has a recording device
with the at least one sensor or the least one interface for a sensor for recording measurements of the at least one vital function of the animal and/or
with the at least one sensor or the at least one interface for a sensor for recording measurements of the movement behavior of the animal,
wherein as an evaluation means the apparatus has a program-controlled processor with a memory, wherein in the memory mappings are stored, in which specific audio files and/or control instructions are assigned to specific measurements or measurement combinations,
wherein the program-controlled processor activates the playback device by means of defined control commands for playing back audio files determined from the mappings.

3. The apparatus as claimed in claim 2, wherein by means of the mappings, measurements or measurement combinations are assigned to loudness specifications as control instructions.

4. The apparatus as claimed in claim 2, wherein
the sensor or sensors of the recording device), which are used for recording measurements of the at least one vital function, is or are implemented as a heart-rate sensor and/or a respiration frequency sensor and/or a body temperature sensor and/or a moisture sensor and/or a blood pressure sensor and/or a thermal imaging sensor and/or an acoustic sensor and/or an acceleration sensor and/or
the sensor or sensors of the recording device, which are used for recording measurements of the movement behavior, is or are implemented as an image sensor and/or as a movement sensor and/or as an acceleration sensor.

5. The apparatus as claimed in claim 1, wherein
the apparatus comprises as an interface at least one network module for the wireless connection of at least one of the sensors and/or for the wireless integration of the apparatus into a wireless network, wherein the wirelessly connected sensor is connected to the animal by means of a belt or is implanted in the animal and/or
the apparatus comprises as an interface at least one network module for the wired connection of at least one of the sensors and/or the wired integration of the apparatus into a wired network.

6. The apparatus as claimed claim 1, wherein
the output device comprises at least two speakers, wherein the speakers have adjacent frequency spectra and/or partially overlapping frequency spectra and/or identical frequency spectra,
wherein the output device comprises at least one contact loudspeaker which is matched to a low frequency spectrum and/or the output device comprises at least one tweeter which is matched to a high frequency spectrum,
wherein at least one of the speakers is designed as an active loudspeaker, which is wirelessly connected to the playback device.

7. The apparatus as claimed claim 2, wherein the recording device comprises at least one additional sensor for detecting a person approaching the animal or for detecting a person present in the vicinity of the animal, wherein the recording device comprises an image sensor and/or a motion sensor, wherein the program-controlled processor is programmed in such a way that, in particular using face recognition and/or gesture recognition, it can distinguish people with access authorization and people without access permission, and the apparatus comprises an alarm system, wherein the alarm system comprises an optical and/or acoustic alarm output unit and/or an alarm relay unit connected to a wired or wireless network.

8. The apparatus as claimed in claim 1, wherein the recording device comprises at least one additional sensor for recording a function of technical equipment of an animal stall, wherein one of the additional sensors is implemented as a mass flow sensor for a supply pipe of a drinking trough and/or wherein one of the additional sensors is implemented as a monitoring sensor for a door or a window or a feeding flap.

9. The apparatus as claimed in claim 2, wherein at least one of the audio files comprises sounds produced by animals, and preferably grazing sounds of ungulates.

10. The apparatus as claimed in claim 1, wherein
as an evaluation means the apparatus comprises a program-controlled processor with a memory and at least one additional sensor that generates measurements for environmental parameters, wherein in the memory mappings are stored, in which specific control instructions are assigned to specific measurements or measurement combinations,
the apparatus comprises a control device and that the program-controlled processor controls the latter using defined control commands for monitoring devices for influencing environmental parameters, wherein the devices are implemented as a lighting device and/or as a fragrance dispenser device and/or a cooling device and/or a heating device.

11. A system for monitoring and/or controlling at least two animals, wherein the system comprises at least two sets of apparatus as claimed in claim 1, wherein the apparatuses are connected to each other via a communication link in such a way that each apparatus has access to the current measurements of the other apparatus or apparatuses and/or to the control commands of the other apparatus or apparatuses for playing individual audio files.

12. A method for monitoring and/or controlling an animal by repeatedly recording at least one vital function and/or a movement behavior of the animal by means of an apparatus, comprising the steps:
storing audio files are stored in a means for storing audio data,
using recording means which are implemented as a recording device and comprise at least one sensor and/or at least one interface for a sensor for recording measurements of the at least one vital function of the animal and/or which comprise at least one sensor and/or at least one interface for a sensor for recording measurements of the movement behavior of the animal, for recording at least one measurement,
using an evaluation means that comprises a program-controlled processor with a memory, wherein in the memory mappings are stored, by means of which one of the stored audio files is identified, which is assigned to a specific measurement or specific measurements, and
playing individual audio files from commands defined by the program-controlled processor on a playback device by activating an output device.

13. The method as claimed in claim 12, wherein
while an audio file is being played, the current measurements recorded by the recording device are compared by means of the program-controlled processor with older measurements recorded by the recording device, and evaluated by the program-controlled processor as to whether an improvement in the vital functions and/or the movement behavior is obtained by playing one of the audio files,
wherein in the event of a deterioration in the vital functions and/or the movement behavior, a message is generated by the program-controlled processor, which is forwarded via a network module of the apparatus to at least one message receiver,
wherein the message receiver is implemented as a mobile phone and/or an alarm device.

14. The method as claimed in claim 12, wherein
the recording device comprises at least one additional sensor for detecting a person approaching the animal or for detecting a person (M)-present in the vicinity of the animal,
wherein the recording device comprises an image sensor and a motion sensor,
wherein the software-controlled processor is programmed with distinguishing features of persons with access authorization in such a way that persons with access authorization are distinguished from persons without access authorization,
wherein whenever a person without access authorization is detected, the software-controlled processor initiates at least one action,
wherein by means of a first action a video recording of activities of the person without access permission is performed and/or wherein by means of a second action an alarm system of the apparatus is activated and/or wherein by means of a third action of the software-controlled processor a message is generated, which is forwarded via a network module of the apparatus to at least one message receiver, wherein the message receiver is implemented as a mobile phone and/or alarm output device.

15. The method as claimed in claim 12, wherein at least one of the audio files is generated by an audio recording of sounds identical to those occurring in nature, wherein the sounds are generated in a sound spectrum audible to animals and outside a range audible to animals, and wherein the sounds are produced by at least one animal.

16. The method as claimed in claim 12, wherein the sounds from the audio file before they are reproduced are decomposed into different frequency ranges by means of a spectral decomposition and the different frequency ranges are reproduced on different audio output devices and/or on different interfaces for output devices.

17. The method as claimed in claim 12, wherein
as an evaluation means the apparatus comprises a program-controlled processor with a memory and at least one additional sensor that generates measurements for environmental parameters, wherein in the memory mappings are stored, in which specific control instructions are assigned to specific measurements or measurement combinations,
that the apparatus comprises a control device and that by means of defined control commands of the program-controlled processor, devices for influencing environmental parameters are controlled, wherein the devices are implemented as a lighting device and/or as a fragrance dispenser device and/or a cooling device and/or a heating device.
